SEQUENCE LISTING (4 sequences)

SEQ ID NO: 1 - DNA, 600 bp, Artificial Sequence
  Prunus mume
atatcactcttctattcggaatattttctggcccaaatcttcgggctccgccgatgaccacttttccattagagccatttttaaccattagccgccacctctcttcaccagcaagcaacgtcccattatccatcatcttatttatgagtaacttttgtgaccacaaaataaaagcttattattataagtattttatacacatttgcggtcatatagtagcaatggattcaattgatcaatgtcaagtgtattatctagctcttaatttaatggtgtgtttctttctaacgagatttttcctcaacttatgaaatgaggttagtttgggcaacggactgtgtttgacaagcatggactggcggacccagaatttttttagcggaggtgcaatattgactaagtatgaatatggtttttcggaataatcattttctcaagataatttttggcggcttttattccttatacatcaaataagaaaacttgattttatgggattttagtatgaagtatatattgacttaatgagctatcatttttagcctaaatcgtattttgcactaaaaccgaattttcatatttttatttggtgggaatttg

SEQ ID NO: 2 - DNA, 30 bp, Artificial Sequence
  Prunus mume
acgttggatgtgcttgtcaaacacagtccg

SEQ ID NO: 3 - DNA, 31 bp, Artificial Sequence
  Prunus mume
acgttggatgggtgtgtttctttctaacgag

SEQ ID NO: 4 - DNA, 23 bp, Artificial Sequence
  Prunus mume
actaacctcatttcataagttga

(19) United States
(12) Patent Application Publication
(10) Pub. No.: US 2020/0407806 A1
(43) Pub. Date: Dec. 31, 2020

(54) SNP MOLECULAR MARKER TIGHTLY LINKED TO WEEPING TRAIT OF MEI AND DETECTION METHOD AND USE THEREOF

(71) Applicant: BEIJING FORESTRY UNIVERSITY, Beijing (CN)

(72) Inventors: Qixiang Zhang, Beijing (CN); Xiaokang Zhuo, Beijing (CN); Tangchun Zheng, Beijing (CN); Tangren Cheng, Beijing (CN); Jia Wang, Beijing (CN); Lidan Sun, Beijing (CN)

(21) Appl. No.: 16/975,651

(22) PCT Filed: Jan. 30, 2019
(86) PCT No.: PCT/CN2019/073921

§ 371 Date: Aug. 25, 2020

(30) Foreign Application Priority Data
Dec. 10, 2018 (CN) 2018115049682

(51) Int. Cl. C12Q 1/6895

(52) U.S. Cl. CPC C12Q 1/6895; C12Q 2600/156; C12Q 2600/13

(57) ABSTRACT

The invention relates to an SNP molecular marker tightly linked to weeping trait of Mei (Prunus mume Sieb.et Zucc.), and a detection method and use thereof. The present invention provides an SNP molecular marker tightly linked to weeping trait of Mei, which is located at 11182911 bp of chromosome 7 of Mei with a polymorphism of A/C. The present invention develops an SNP molecular marker tightly linked to weeping trait of Mei through the combination of GWAS analysis with the population selection method. The SNP molecular marker has good reproducibility and high accuracy in the identification of weeping/upright traits in the segregation population of filial generation of Mei, and achieves an accuracy of 96% or more in the single-molecule marker identification of weeping/upright traits. The SNP molecular marker tightly linked to weeping trait of Mei and detection method thereof provided by the present invention may realize the selection of weeping/upright traits at the seedling stage, greatly shorten the breeding cycle, improve the breeding efficiency, and reduce the breeding cost, and may be used in practice for molecular-assisted selection breeding of Mei,

### CROSS-REFERENCE TO RELATED APPLICATION

This application claims the priority to the Chinese Patent Application No. 201811504968.2 entitled “SNP MOLECULAR MARKER TIGHTLY LINKED TO WEEPING TRAIT OF MEI AND DETECTION METHOD AND USE THEREOF” filed on Dec. 10, 2018, the disclosure of which is incorporated herein by reference in its entirety.

### TECHNICAL FIELD

The present invention relates to the fields of molecular biology and plant molecular breeding, and specifically, to an SNP molecular marker tightly linked to weeping trait of Mei, and a detection method and use thereof.

### BACKGROUND ART

Mei (Prunus mume Sieb.et Zucc.) belongs to family Rosaceae genus Prunus and is one of the ten famous traditional flowers in China. Mei has ornamental and use values, is originated from southwest China, and has a history of introduction and cultivation for more than 3,000 years (Chen Junyu. China Mei Flower Cultivars [M]. China Forestry Publishing House, 2010). There are various varieties of Mei with plenty of flower colors, flower types and plant architecture. Among them, weeping Mei (Prunus mume var pendula) is one of the nine cultivars with unique plant architecture of Mei. Because of its unique tree posture and other ornamental traits such as flower color, floral scent and the like, weeping Mei is popular with people and has an important position in gardening.

A large number of SNP markers are developed by high-throughput sequencing technology, and the developed trait-linked molecular markers are used for the initial screening of plants so as to achieve the purpose of molecular marker-assisted breeding, which may greatly shorten the breeding cycle of Mei and improve breeding efficiency.

The present invention uses GWAS and population selection method to develop a trait-marker-associated SNP marker, obtains an SNP marker tightly associated with weeping trait of Mei, enables early screening of the target trait, greatly shortens the breeding cycle, improves the breeding efficiency, reduces the breeding cost, and realizes the molecular marker-assisted selection breeding of weeping trait of Mei.

### SUMMARY OF THE INVENTION

In order to solve the technical problems existing in the prior art, the object of the present invention is to provide an SNP molecular marker tightly linked to weeping trait of Mei, and a detection method and use thereof.

In the present invention, 214 cultivars of Mei were subjected to whole-genome sequencing, and the GWAS analysis method was used to detect loci that are significantly associated with branch traits of Mei. Through population selection analysis, it was observed whether there was a selection signal in the area of significantly associated loci; through combination of GWAS association analysis with population selection analysis, 47 SNP markers that were significantly associated with weeping trait of Mei were obtained. The weeping trait was mapped to two intervals of 11.1-11.8 Mb and 14.2-15.2 Mb of chromosome 7 of Mei. The F1 segregation population obtained by hybridization of “LIU BAN” Mei and the “FEN TAI weeping” Mei was used as the material. The 47 SNP markers that were significantly associated were verified by mass spectrometry. It was found that the SNP marker Pm7_11182911 was most tightly associated with the weeping trait of Mei.

Firstly, the present invention provides an SNP molecular marker tightly linked to weeping trait of Mei, which is located at 11182911 bp of chromosome 7 (Prunus mume Genome v1.0 Genbank accession number: PRJNA171605; published on Feb. 28, 2013), and the polymorphism of the SNP molecular marker is A/C.

The above A/C mutation located at 11182911 bp of chromosome 7 of Mei is tightly linked to the weeping/upright traits of Mei. Therefore, all DNA molecular markers including the SNP locus at 11182911 bp of chromosome 7 of Mei (For example, the DNA molecule with the sequence represented by SEQ ID NO. 1, the above-mentioned SNP locus is located at the 300^{th} position of SEQ ID NO. 1) are all within the protection scope of the present invention.

Based on the development of the above-mentioned SNP molecular marker, the present invention has developed an identification method of genotype for weeping/upright traits of Mei. By analyzing the base sequence at 11182911 bp of chromosome 7 of Mei, the weeping/upright traits of Mei are identified: the genotype of the above SNP locus corresponding to weeping trait of Mei is AC, and the genotype corresponding to upright trait of Mei is CC.

To detect the SNP genotype, the present invention provides primers for detection of

SNP molecular marker, and the primers for detection include:

forward primer F:
5′- ACGTTGGATGTGCTTGTCAAACACAGTCCG-3′;
reverse primer R:
5′-ACGTTGGATGGGTGTGTTTCTTTCTAACGAG-3′.

In the present invention, when the genotype of the SNP is analyzed by analyzing the sequence of the product of the single base extension reaction, the primers for detection further include a primer for single base extension:

the primer for single base extension is: 5′-ACTAACCTCATTTCATAAGTTGA-3′.

On this basis, the present invention provides a kit for detection of weeping trait of Mei, including a combination of the above-mentioned forward primer F and the reverse primer R or including a combination of the above-mentioned forward primer F, the reverse primer R and the primer for single base extension.

In addition, the kit further includes dNTPs, DNA polymerase, Mg²⁺, and PCR reaction buffer.

Preferably, the kit according to the present invention further includes standard positive template and SAP enzyme.

Further, the present invention provides the use of the SNP molecular marker or the primers for detection or the kit in the identification of weeping trait or upright trait of Mei.

The present invention also provides the use of the SNP molecular marker or the primers for detection or the kit in molecular-assisted breeding of Mei.

Further, the present invention provides a method for identifying plant type trait of Mei, including: performing a PCR amplification using a forward primer F and a reverse primer R with the genome of Mei to be tested as a template, analyzing the sequence of the product of the PCR amplification, and judging the genotype of Mei to be tested;

the sequence of the forward primer F is 5′-ACGTTGGATGTGCTTGTCAA ACACAGTCCG-3′; and the sequence of the reverse primer R is 5′-ACGTTGGATGGGTGTGTTTCTTTCTAACGAG-3′.

In the present invention, the product of the PCR amplification obtained by the PCR amplification described above may be subjected to sequence analysis using a conventional SNP typing method in the art, such as a direct sequencing method, a mass spectrometry detection method and the like.

In order to better meet the requirements for SNP typing of high-throughput, high-precision, high-efficiency, low-cost, and broad-spectrum analysis, preferably, the method of analyzing the sequence of the amplified product is a SNP genotype analysis using Sequenom MassARRAY®SNP technology, and includes the following steps:

(1) treating the product of the PCR amplification with alkaline phosphatase;

(2) performing a single base extension reaction using the alkaline phosphatase-treated product obtained in step (1) as a template, and the sequence of the primer for the single base extension reaction is: 5′-ACTAACCTCATTTCATAAGTTGA-3′; and

(3) analyzing the genotype of the product of the single base extension reaction.

The genotype of the product of the single base extension reaction is analyzed using Sequenom MassARRAY®SNP technology and mass spectrometric detection.

In order to better meet the requirements of high-throughput and automated analysis, as a preferred embodiment of the present invention, the method for identifying plant type traits of Mei includes the following steps:

1) extracting genomic DNA of Mei plant to be tested;

2) performing a PCR amplification reaction using the forward primer F and the reverse primer R with genomic DNA of the plant to be tested as a template;

(3) treating the PCR product with alkaline phosphatase to remove free dNTPs from the system;

(4) performing a single base extension reaction using a primer for the single base extension reaction: 5′-ACTAACCTCATTTCATAAGTTGA-3′ with the alkaline phosphatase-treated product as a template;

(5) using resin to purify the extension product after the extension reaction is completed; and

(6) transferring the extension product to a MassARRAY SpectroCHIP chip, detecting by a MassARRAY Analyzer ComPmc mass spectrometer, and analyzing the results of mass spectrometric detection by software to obtain typing data of the SNP locus.

In the above step (2), the reaction system (5 μL) of the PCR amplification reaction is as follows:

0.625 μL of 10×PCR Buffer (containing Mg²⁺), 0.325 μL of 25 mM MgCl₂, 1.0 μL of dNTP (2.5 mM each), 0.5 μL of forward primer F, 0.5 μL of reverse primer R, 0.1 μL of 5 U/μL Taq DNA polymerase, 1.0 μL of genomic DNA template (50 ng/μL), supplemented with water to 5 μL.

The reaction procedure is as follows: 94° C. for 15 min; 45 cycles of 94° C. for 10 to 20 sec, 56 to 65° C. for 30 sec, 72° C. for 1 min; and 72° C. for 3 to 5 min.

In the above step (3), the reaction system (7 μL) of alkaline phosphatase treatment is as follows: 0.17 μL of 10×SAP Buffer, 0.3 μL of 1 U/μL SAP enzyme, 5 μL of the PCR product from step (2), supplemented with water to 7 μL.

The reaction procedure is as follows: 37° C. for 40 min; and 85° C. for 5 min. In the above step (4), the reaction system (9 μL) of the single base extension reaction is as follows: 0.2 μL of 10×iPlex Buffer, 0.2 μL of iPlex Termination mix, 0.804 μL of primer for the single base extension, 0.041 μL of iPlex enzyme, 7 μL, of the alkaline phosphatase-treated product obtained from step (3), supplemented with water to 9 μL.

The reaction procedure is as follows:

| 94° C. | 30 sec | | | | |
|---|---|---|---|---|---|
| 94° C. | 5 sec | | | | |
| 52-56° C. | 5 sec | | 5 cycle | | 40 cycle |
| | | {close oversize brace} | | {close oversize brace} | |
| 80° C. | 5 sec | | | | |

| | |
|---|---|
| 72° C. | 3 min |
| 4° C. | forever. |

The present invention has the following beneficial effects:

(1) The present invention combines GWAS analysis and population selection method to develop an SNP molecular marker Pm7_11182911 that is tightly linked to weeping trait of Mei. The SNP molecular marker has the advantage of good reproducibility and high accuracy in the identification of weeping/upright traits in the segregation population of filial generation of Mei, and realizes a detection effect of the identification accuracy rate of single molecule marker of 96% or more.

(2) The present invention provides an efficient detection method for the SNP molecular marker Pm7_11182911, which has the advantages of high throughput, low detection cost, and no influence from environmental conditions.

(3) The molecular marker Pm7_11182911 and the detection method provided by the present invention may realize the selection of weeping/upright traits at the seedling stage, greatly reduce the breeding workload, significantly shorten the breeding cycle of new cultivars of weeping Mei, improve the breeding efficiency, reduce the breeding cost, and may be used in practice for molecular-assisted selection breeding of Mei.

### BRIEF DESCRIPTION OF THE DRAWINGS

FIG. 1 is a schematic diagram of a measuring method for phenotypic traits in Example 2 of the present invention, in which panel (a) is branch angles during growing period; panel (b) is branch angles during mature period; and panel (c) is angles between different parts of mature branches and the back direction of gravity.

FIG. 2 is a Manhattan diagram of GWAS analysis in Example 2 of the present invention, in which panels (a)-(g) are GWAS analysis results of the branch angle A1 during growing period, the branch angle A2 during the mature period, the angle T1 between different parts of the branch and the antigravity direction, the angle T2 between different parts of the branch and the antigravity direction, the angle T3 between different parts of the branch and the antigravity direction, the angle T4 between different parts of the branch and the antigravity direction, and the angle T5 between different parts of the branch and the antigravity direction, respectively.

FIG. 3 is a population selection analysis diagram of Example 3 of the present invention, in which panel (a) is the coefficient of genetic differentiation among populations (FsT); panel (b) is the nucleic acid diversity (Pi) of weeping Mei; panel (c) is the nucleic acid diversity (Pi) of upright Mei; and panel (d) is enlarged drawings of the analysis results of the coefficient of genetic differentiation among populations and the nucleic acid diversity.

FIG. 4 is marker-trait-associated loci screened by the combination of the population selection and GWAS in Example 3 of the present invention, in which panel (a) is associated loci screened by the combination; and panel (b) is comparison of allele frequency and nucleic acid diversity of candidate associated loci.

FIG. 5 is diagrams of the results of mass spectrometry analysis in Example 4 of the present invention, in which panel (a) is peak patterns of the AC genotype of the SNP marker Pm7_11182911, and panel (b) is peak patterns of the CC genotype of the SNP marker Pm7_11182911.

FIG. 6 is analysis diagrams of the typing results of the SNP marker Pm7_11182911 in Example 4 of the present invention, in which panel (a) is a genotype frequency analysis of upright Mei and weeping Mei; and panel (b) is a scatter diagram of genotypes.

### SPECIFIC MODES FOR CARRYING OUT THE EMBODIMENTS

Specific modes for carrying out the embodiments of the present invention will be further described in detail in combination with Examples. It should be understood that the following Examples are given for illustrative purposes only, and are not intended to limit the scope of the present invention. A person skilled in the art can make various modifications and substitutions to the present invention without departing from the aim and spirit of the present invention.

Unless otherwise specified, the experimental methods used in the following Examples are conventional methods.

Unless otherwise specified, the materials, reagents and the like used in the following Examples are all commercially available.

### EXAMPLE 1

### Development of SNP Markers for Whole Genome of Mei

### 1. Experimental Materials

The test materials were 214 cultivars of Mei, which were collected from Wuhan Moshan Germplasm Resource Nursery of Mei (30.546719° N, 114.413254° E), and included conventional cultivars from 11 regions including Hubei, Yunnan, Jiangsu, and Anhui (such as cultivar groups of GONG FEN, ZHU SHA, DAN BAN, YU DIE, XING MEI, HUANG XIANG, CHUI ZHI, TIAO ZHI and LONG YOU).

### 2. Extraction of Genomic DNA

The materials for genomic extraction of 214 cultivars of Mei were selected from fresh leaves or silica gel-dried leaves. Genomic DNA was extracted according to the CTAB (Cetyl trimethyl ammonium bromide) method. A 1.0% agarose gel was prepared. 3 μL of genomic DNA was suck up and mixed with about 1 μL of loading-buffer, and then the samples were loaded into the loading well. Electrophoresis was performed at 150 V for 15 minutes to detect the integrity of the genomic DNA. The concentration and purity of the genomic DNA were measured with NANO DROP 2000, so as to ensure that the concentration of the genomic DNA was >50 ng/μL. The DNA concentration was accurately quantified using Qubit. Qualified DNA samples were used for library sequencing.

### 3. Library Sequencing

According to the requirements of the Illumina sequencing platform, the qualified DNA samples were randomly interrupted. The DNA fragments were subjected to steps of end repair, addition of PolyA tail, addition of sequencing adapter, purification, PCR amplification and library detection. The short insertion segment was 500 bp and the long insert was 2 Kb in the libraries. After all libraries passed the test, they were sequenced using the Illumina HiSeq200 sequencing platform.

### 4. Quality Control Test

The rawdata was filtered based on the following 5 criteria:

(1) the number of mismatch bases in the reads is greater than 10%; (2) reads are low-quality sequences (Phred quality scores Q the number of bases of short insertion segments are more than 65%, the number of bases of long insertion segments are more than 80%); (3) adapter sequence of 10 bp; (4) overlapping of short insertion segments with more than 10 bp at both ends; (5) reads with identical double-ended sequence. After strict filtering of the sequencing data, the data that meets the above 5 criteria was removed to obtain high-quality clean data for subsequent analysis.

### 5. SNPs Detection and Quality Control

The high-quality sequencing data was compared to the reference genome by BWA software, and SNP calling and quality control were performed using GATK software (quality control parameters: QD<2.0∥FS>60.0∥MQ<40.0∥ HaplotypeScore>13.0). At the same time, SNPs with genotype loss of more than 10% were filtered, SNP loci with a minimum allele frequency (MAF) of less than 0.01 were removed, SNP loci with Hardy-Weinberg detection P<10⁻⁶ were removed, triple or multiple alleles were removed, only double alleles were retained. In the end, 3,014,409 high-quality SNP loci were obtained for subsequent GWAS analysis.

### EXAMPLE 2

### Mining of SNP Markers Associated with Weeping Trait of Mei Based on GWAS Analysis

### 1. Phenotype Determination of Plant Type Traits of Mei

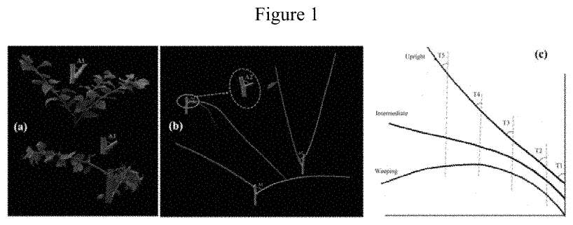

The phenotypic traits of 214 cultivars of Mei were collected. The collection method of phenotypic traits was shown in FIG. 1. The branch angles of mature branches and growing branches were measured. At the same time, mature branches were divided into five parts. The angle between each part and the antigravity direction was measured. A total of 7 phenotypic traits were obtained for GWAS analysis.

### 2. Mining of Traits-Markers Associated Loci by GWAS

The ADMIXTURE1.3 software was used to calculate matrix files (Q) of the population structure of 214 cultivars of Mei, and the SPMGeDi v.1.4b software was used to calculate the kinship matrix (K). The TASSEL v.5 software was used to perform GWAS analysis, and a mixed linear model (MLM) was selected. The model includes fixed effects, random effects, and genetic relationships, as follows:

y=Xβ+Zμ+e

in which, y is a phenotype, β is a marker and Q, μ is genetic effect, X and Z are known matrices, and e is residual.

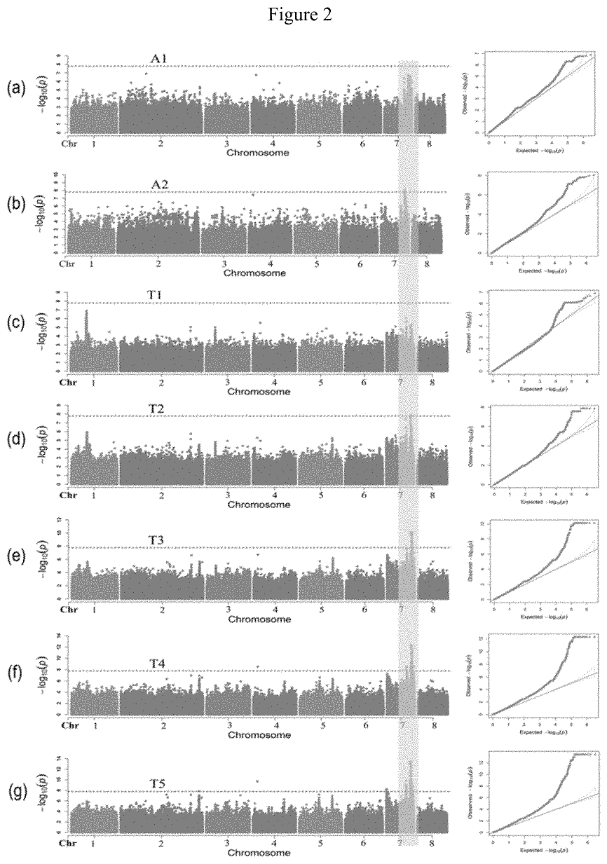

Using GWAS association analysis, in all seven phenotypic traits, loci that are significantly associated with weeping traits were detected on the Pm7 chromosome (FIG. 2). The association regions were located in the regions of 11-12 Mb and 14-16 Mb.

### EXAMPLE 3

### Mining of Selected Loci for Weeping Traits of Mei Based on Population Selection Analysis

### 1. Population Selection Analysis

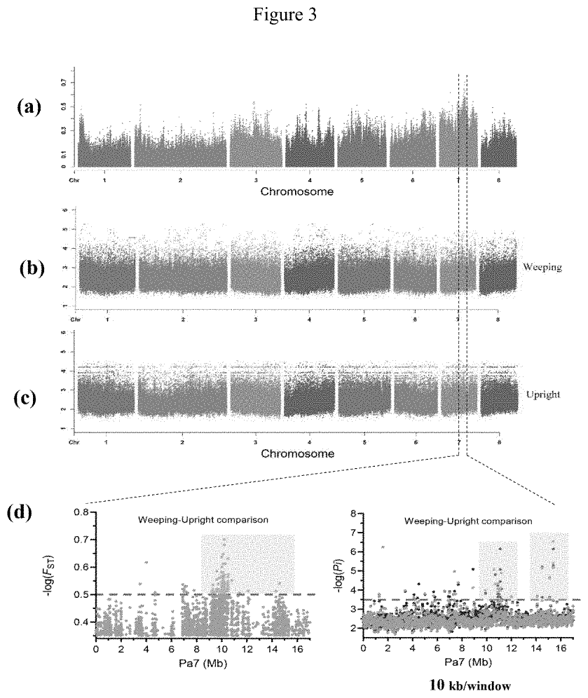

The VCFtools v. 0.1.15 software was used to analyze the coefficient of genetic differentiation among populations (FsT) of weeping Mei and upright Mei and the nucleic acid diversity (Pi). It was found that the regions of 10 to 12 Mb and 14 to 15.5 Mb of Pm7 were strongly selected (FIG. 3). This result was the same as the GWAS results, further confirming that SNP marker located in this region is tightly associated with weeping trait.

### 2. Combination of Population Selection and GWAS for Screening Candidate Markers

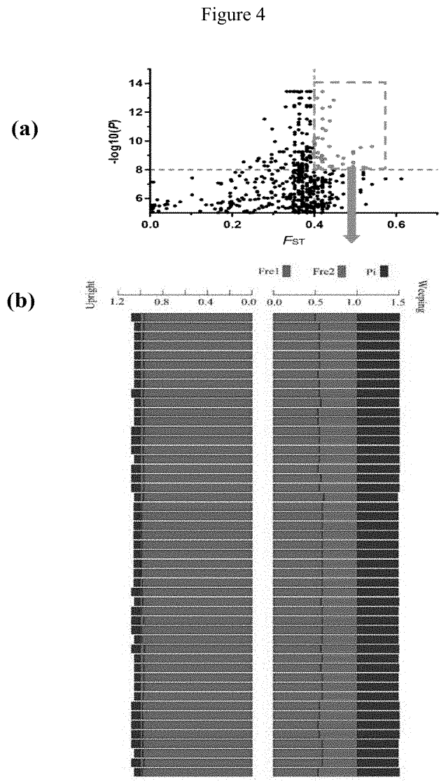

Based on the combination of results of population selection analysis and GWAS analysis, 49 SNP markers tightly associated with weeping trait of Mei were screened out. When comparing the allele frequency and nucleic acid diversity of these 49 SNP loci, it was found that there were significant differences between weeping Mei and upright Mei (FIG. 4). The combination of population selection and GWAS selection greatly reduced the number of candidate markers and increased the reliability of SNP markers.

### EXAMPLE 4

### Verification of Candidate SNP Loci

### 1. Experimental Materials

The F1 segregation population obtained by hybridization of “LIU BAN” Mei as the female parent and “FEN TAI weeping” Mei as the male parent was used as the material, of which 127 were weeping individuals and 161 were upright individuals. The experimental materials were planted in He Village, Moganshan Town, Huzhou City, Zhejiang Province (30.566389° N, 119.879582° E).

### 2. Extraction of Genomic DNA

Extraction of genomic DNA was performed according to the instructions of the High-Efficiency Plant Genomic DNA Extraction Kit (Tiangen Biochemical Technology Co., Ltd.). A 1.0% agarose gel was prepared. 3 μL of DNA was suck up and mixed with about 1 μL of loading-buffer. Electrophoresis was performed at a voltage of 150 V for 15 min to detect the integrity of DNA. DNA concentration and purity were determined with NANO DROP 2000, so as to ensure that the DNA concentration was >50 ng/μL. Qualified DNA samples were used for mass spectrometry detection experiments.

### 3. Primer Design

Based on the position information of SNP, the 47 sequences with 300 bp before and after the SNP loci were extracted, and primers for PCR and single-base extension reactions were designed. The nucleotide sequence of the Pm7_11182911 locus was represented by SEQ ID NO. 1. According to the sequence information, the Genotyping Tools and MassARRAY Assay Design software were used to design primers for PCR amplification and single base extension for the SNP loci to be tested. The sequences of amplification primers for the Pm7_11182911 locus were as follows:

Primer 1 for PCR amplification:
ACGTTGGATGTGCTTGTCAAACACAGTCCG;
Primer 2 for PCR amplification:
ACGTTGGATGGGTGTGTTTCTTTCTAACGAG;
primer for single base extension:
ACTAACCTCATTTCATAAGTTGA.

### 4. PCR Amplification

The multiplex PCR amplification technology was performed in a 384-well plate. The reaction system of each well was 5 μL. The reaction system was shown in Table 1.

TABLE 1
PCR reaction system

| Name of reagents | Each reaction (4) |
|---|---|
| H₂O | 0.95 |
| PCR Buffer (10×, with 15 mM MgCl₂) | 0.625 |
| MgCl₂ (25 mM) | 0.325 |
| dNTP (2.5 mM each) | 1.0 |
| Working solution of primers | 1.0 |
| HotstarTaq (5 U/μL) | 0.1 |
| DNA template | 1.0 |
| Final volume | 5.0 |

The PCR reaction procedure was as follows:

| 94° C. | 15 min; | | | |
|---|---|---|---|---|
| | 94° C. | 20 sec | | |
| | 56° C. | 30 sec | {close oversize brace} | 45 cycles; |
| | 72° C. | 1 min | | |

| | |
|---|---|
| 72° C. | 3 min; |
| 4° C. | forever. |

### 5. Alkaline Phosphatase Treatment to PCR Product (SAP Treatment)

After the PCR reaction was completed, the PCR product were treated with SAP to remove free dNTPs. The SAP reaction solution was as follows:

TABLE 2
Formulation of SAP reaction solution

| Name of reagents | Each reaction (μL) |
|---|---|
| H₂O | 1.53 |
| SAP Buffer (10×) | 0.17 |
| SAP enzyme (1 U/μL) | 0.3 |
| Final volume | 2.0 |

For each reaction well of alkaline phosphatase treatment, the total volume of the reaction system was 7 μL, of which 5 μL was the PCR product, and 2 μL was the SAP mixture. The reaction procedure was as follows: 37° C., 40 min; 85° C., 5 min; and 4° C., forever. After setting the program, the PCR instrument was started for alkaline phosphatase treatment.

### 6. Single Base Extension

After the alkaline phosphatase treatment was completed, a single base extension reaction was performed. The reaction solution system was as follows:

TABLE 3
Formulation of alkaline phosphatase reaction solution

| Name of reagent | Each reaction (μL) |
|---|---|
| H₂O | 0.755 |
| iPlex Buffer (10×) | 0.2 |
| iPlex Termination mix | 0.2 |
| Working solution of primers | 0.804 |
| iPlex enzyme | 0.041 |
| Final volume | 2.0 |

For each reaction well, the single-base extension reaction system was 7 μL of the PCR product after SAP treatment and 2 μL of the extension reaction solution, and the total volume of the reaction system was 9 μL. The 384-well plate into which the extension reaction system had been added was put into the PCR instrument, and the reaction procedure named “extension” was run. The reaction procedure was as follows:

| 94° C. | 30 sec | | | | |
|---|---|---|---|---|---|
| 94° C. | 5 sec | | | | |
| 52° C. | 5 sec | | 5 cycle | | 40 cycle |
| | | {close oversize brace} | | {close oversize brace} | |
| 80° C. | 5 sec | | | | |

| | |
|---|---|
| 72° C. | 3 min |
| 4° C. | forever. |

### 7. Purification of Product

6 mg of resin was taken and evenly covered on a 384-well resin scraper. The excess resin was scraped off, and the scraper was left for 20 min. After the reaction was completed, the 384-well plate was centrifuged at 1000 rpm for 1 min. 25 μL of deionized water was added to each well. The 384-well plate was placed on the resin plate upside down (pay attention to the fixation and it cannot be shifted). Then the resin plate was inverted on the 384-well plate. The resin plate was knocked such that the resin was dropped into the 384-well plate and the 384-well plate was sealed with film. The long axis of the 384-well plate was taken as the axis, and the 384-well plate was flipped for 20 minutes, and then was centrifuged at 3500 rpm for 5 minutes for later use.

### 8. Detection

The detection steps of the product obtained in step (7) were as follows:

(1) spotting on the Nanodispenser SpectroCHIP chip. The sample for detection was transferred from the 384-well reaction plate to a MassARRAY SpectroCHIP chip covered with a substrate;

(2) MassARRAY Analyzer ComPmc mass spectrometry detection. After the sample was transferred to the SpectroCHIP chip, the chip may be put into the mass spectrometer for detection. Each sample on the chip was detected for 3 to 5 sec, and the detection process was fully automated;

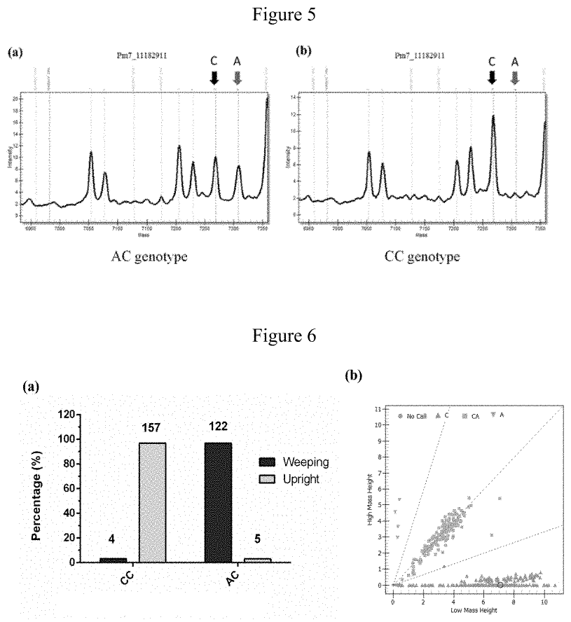

(3) The experimental results were analyzed by TYPER software to obtain typing data. The results of mass spectrometry typing were shown in FIG. 5.

### 9. Results Analysis

The genotype frequency of the F1 segregation population obtained by hybridization of “LIU BAN” Mei as the female parent and “FEN TAI weeping” Mei as the male parent was calculated using the results of mass spectrometry typing. The difference of genotype frequency between the upright individuals and the weeping individuals at SNP locus Pm7_11182911 was the largest, which may accurately separate weeping individuals from upright individuals. The 161 upright individuals and 127 weeping individuals in the F1 population was subjected to typing, and the accuracy rates were 97.5% (157/161) and 96.7% (122/127), respectively. The upright individuals showed a CC genotype and the weeping individuals showed an AC genotype (FIG. 6).

The present invention establishes a molecular marker-assisted selection breeding system for weeping Mei, which can achieve early selection of weeping trait by using single marker, greatly improves breeding efficiency, reduces breeding workload, field planting area and labor, greatly reduces breeding costs and cycle, and has groundbreaking value for molecular-assisted selection breeding of weeping Mei.

The above is only a preferred embodiment of the present invention. It should be noted that for those of ordinary skill in the art, without departing from the technical principles of the present invention, several improvements and modifications may be made. These improvements and modifications should also be regarded as the protection scope of the present invention.

### INDUSTRIAL APPLICABILITY

The present invention provides an SNP molecular marker tightly linked to weeping trait of Mei, which is located at 11182911 bp of chromosome 7 of Mei with a polymorphism of A/C. The present invention develops an SNP molecular marker tightly linked to weeping trait of Mei through the combination of GWAS analysis with population selection. The SNP molecular marker has good reproducibility and high accuracy in the identification of weeping/upright traits in the segregation population of filial generation of Mei, and achieves an accuracy of 96% or more in the single-molecule marker identification of weeping/upright traits. The SNP molecular marker tightly linked to weeping trait of Mei and detection method thereof provided by the present invention may realize the selection of weeping/upright traits at the seedling stage, greatly shorten the breeding cycle, improve the breeding efficiency, and reduce the breeding cost, and may be used in practice for molecular-assisted selection breeding of Mei, which has good economic value and application prospects.

What is claimed:

1. An SNP molecular marker tightly linked to weeping trait of Her, wherein, the SNP molecular marker is located at 11182911 by of chromosome 7 of Mei, and the polymorphism of the SNP molecular marker is A/C.

2. Primers for detection of the SNP molecular marker according to claim 1, wherein the primers for detection comprise:
forward primer F:
5′-ACGTTGGATGTGCTTGTCAAACACAGTCCG-3′;
reverse primer R:
5′-ACGTTGGATGGGTGTGTTTCTTTCTAACGAG-3′.

3. The primers for detection according to claim 2, wherein the primers for detection further comprise a primer for single base extension:
the sequence of the primer for single base extension is as follows:
5′-ACTAACCTCATTTCATAAGTTGA-3′.

4. A kit for detection of weeping trait of Mei, wherein the kit comprises the primers for detection according to claim 2 or claim 3.

5. The kit according to claim 4, wherein the kit further comprises dNTPs, DNA polymerase, Mg²⁺, and PCR reaction buffer;
preferably, the kit further comprises standard positive template and SAP enzyme.

6. (canceled)

7. (canceled)

8. A method for identifying plant type trait of Mei, comprising: performing a PCR amplification using a forward primer F and a reverse primer R with the genome of Mei to be tested as a template, analyzing the sequence of the product of the PCR amplification, and judging the genotype of Mei to be tested.;
the sequence of the forward primer F is 5′-ACGTTGGATGTGCTTGTCAA ACACAGTCCG-3′; and the sequence of the reverse primer R is 5′-ACGTTGGATGGGTGTGTTTCTTTCTAACGAG-3′.

9. The method according to claim 8, wherein the method of analyzing the sequence of the product of the PCR amplification comprises the following steps:
(1) treating the product of the PCR amplification with alkaline phosphatase;
(2) performing a single base extension. reaction using the alkaline phosphatase-treated product obtained in step (1) as a template, and the sequence of the primer for single base extension reaction is: 5′-ACTAACCTCATTTCATAAGTTGA-3′; and
(3) analyzing the genotype of the product of the single base extension reaction.

10. The method according to claim 9, wherein the genotype of the product of the single base extension reaction is analyzed using Sequenom MassARRAY®SNP technology and mass spectrometric detection.
